(19) United States
(12) Patent Application Publication
(10) Pub. No.: US 2016/0082236 A1
(43) Pub. Date: Mar. 24, 2016

(54) TRANSPLANTATION DEVICE AND METHOD OF USE

(71) Applicant: The Regents of the University of California, Oakland, CA (US)

(72) Inventors: Elliot L. Botvinick, Irvine, CA (US); Steven C. George, Irvine, CA (US); Bhupinder S. Shergill, Stockton, CA (US); Jonathan R. T. Lakey, Irvine, CA (US)

(21) Appl. No.: 14/960,150

(22) Filed: Dec. 4, 2015

Related U.S. Application Data
(63) Continuation of application No. PCT/US2014/041307, filed on Jun. 6, 2014.
(60) Provisional application No. 61/832,602, filed on Jun. 7, 2013.

(51) Int. Cl. A61M 31/00; A61B 5/1459; B29C 65/00; B29C 35/08; B29C 33/42; B29C 65/48; A61B 5/1455; B29C 33/44

(52) U.S. Cl. CPC A61M 31/002; A61B 5/14556; A61B 5/1459; B29C 33/448; B29C 35/0805; B29C 33/42; B29C 65/4805; B29C 66/45; A61M 2202/06; A61M 2205/04; A61M 2207/00; B29C 2035/0827; B29K 2005/00; B29K 2995/0056; B29L 2031/7532

(57) ABSTRACT

Embodiments of the present disclosure relates to an implantable structure and a two stage method for cell and/or tissue transplantation. The implantable structure is configured to promote vascularization prior to cell and/or tissue transplantation, thereby allowing for implanted cells and/or tissues to have increased viability. In some embodiments, oxygen sensitive dyes can be used to determine levels of vascularization of the device.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### BACKGROUND

Diabetes is the 4th leading cause of death in the United States with more than 3 million Americans currently suffering from type 1 diabetes. An average of 80 people per day is diagnosed with the disease, with half of those being children, and presently there is no cure. Current treatments for type 1 diabetes include artificial insulin injection and transplanting tissue containing islets (structures containing insulin secreting beta cells). Both treatments however, have considerable downsides, such as constant monitoring of blood glucose levels when injecting artificial insulin, and lifelong use of immune suppressing drugs with tissue transplantation (which could lead to other infections and cancer since the immune system is compromised).

The primary treatment of type 1 diabetes is the delivery of artificial insulin via injection or pump combined with careful monitoring of blood glucose levels using blood-testing monitors. Transplantation of functional human islet tissue by the “Edmonton Protocol”, a technique pioneered by Dr. Jonathan Lakey, restores euglycemia by replacing islet cells lost to autoimmune destruction. The Edmonton Protocol demonstrates the ability to restore good glycemic control after transplantation. The downside to this process however, is the required use of lifelong pharmaceutical immune suppression which may cause significant side effects, including elevated risk of infections and cancer, making such islet tissue transplantation appropriate only for diabetic patients with life-threatening complications.

Encapsulation of islet tissue, which prevents direct contact with the host's immune system, may allow transplantation without pharmaceutical immune suppression, and may allow use of porcine or other suitable xenograft tissue which is in great supply compared to human. However, the success of microencapsulation has been limited.

### SUMMARY

Disclosed herein are embodiments of a device for cell transplantation comprising a biocompatible frame configured to be inserted into tissue, at least one slit passing through the frame, wherein the at least one slit is sized and configured to allow vascular perfusion through the at least one slit, and a fluidic channel located within the frame and comprising a semipermeable surface region configured to retain cells while allowing certain dissolved molecules to diffuse between the fluidic channel and the at least one slit.

In some embodiments, the device can further comprise at least one inlet/outlet port in fluid communication with the fluidic channel. In some embodiments, the at least one inlet/outlet port can be configured to be sealed.

In some embodiments, the fluidic channel can be configured to retain islet cells.

In some embodiments, the frame can be formed from a plurality of layers bonded together. In some embodiments, the frame can be monolithic.

In some embodiments, the frame can be a hydrogel. In some embodiments, the frame can be formed from a material selected from the group consisting of alginate, polydimethylacrylamide (PDMA), polydimethylsiloxane (PDMS), polyacrylonitrile (PAN) or polymethylmethacrylate (PMMA).

In some embodiments, the device can further comprise a plurality of slits. In some embodiments, the device can further comprise a plurality of fluidic channels. In some embodiments, the semipermeable surface region of the fluidic channel can comprise dialysis tubing. In some embodiments, the biocompatible frame can be hollow and can at least partially define the fluidic channel. In some embodiments, the fluidic channel can be serpentine.

In some embodiments, the device can further comprise an oxygen sensitive dye incorporated into the device. In some embodiments, the oxygen sensitive dye can have a fluorescence lifetime based on oxygen levels.

In some embodiments, one or more agents promoting vascularization can be incorporated into the device. In some embodiments, the one or more agents promoting vascularization can be autologous blood, fibrin purified from donor mice, VEGF, or other growth factors.

Also disclosed herein are embodiments of a method for making an transplantation device for islet transplantation comprising fabricating a bottom layer having at least one slit, fabricating an inner layer having a fluidic channel comprising a semipermeable surface region and an injection port, fabricating a top layer having at least one slit, the at least one slit of the top layer configured to substantially align with the at least one slit on the bottom layer, and bonding the layers together to sandwich the inner layer between the top and bottom layers thereby enclosing the fluidic channel, wherein the fluidic channel is positioned to allow diffusion communication between the fluidic channel and the slits. In some embodiments, the top and bottom layers can comprise a plurality of slits.

Also disclosed herein are embodiments of a method for making a device for islet transplantation comprising preparing a dissolvable mold configured to form an implantable device comprising a biocompatible frame configured to be inserted into tissue, at least one slit configured to pass at least partially through the frame, wherein the at least one slit is sized and configured to promote vascular perfusion, and a fluidic channel configured to retain cells and located within the biocompatible frame, the fluidic channel being separated from the at least one slit and able to communicate with the at least one slit through diffusion, adding a polymerizable material to the mold, polymerizing the material to form the implantable device, and dissolving the mold.

In some embodiments, polymerizing the material can comprise polymerizing the material with UV light. In some embodiments, dissolving the mold can comprise submerging the mold in a dissolving solution. In some embodiments, the dissolving solution can be citrus oil.

Also disclosed herein are embodiments of a method for treating diabetes comprising implanting the device comprising a biocompatible frame configured to be inserted into tissue, at least one slit passing through the frame, wherein the at least one slit is sized and configured to allow vascular perfusion through the at least one slit, and a fluidic channel located within the frame and comprising a semipermeable surface region configured to retain cells while allowing certain dissolved molecules to diffuse between the fluidic channel and the at least one slit into a tissue of a diabetic patient, equilibrating the device within the tissue for a period of time sufficient to allow vascularization of the at least one slit, and injecting a suspension of islet cells into the fluidic channel, wherein the islet cells secrete insulin into the at least one vascularized slit in response to glucose levels in the at least one vascularized slit.

### BRIEF DESCRIPTION OF THE DRAWINGS

FIGS. 1A-F illustrate an embodiment of a transplantation device.

FIGS. 2A-B illustrate the preliminary results showing that embodiments of the transplantation device's slits can be perfused following one week subcutaneous implantation.

FIG. 3 shows a fluidic channel cast in 5% alginate.

FIG. 4 illustrates the process of islet implantation.

FIGS. 5A-D illustrate a generalized view of the approach when using embodiments of the device.

FIGS. 6A-C illustrate an embodiment of a mold and hydrogel.

FIG. 7A-F illustrate an embodiment of the transplantation device.

FIG. 8 illustrates an embodiment of the transplantation device that includes a frame with dialysis tubing and support posts.

FIG. 9 illustrates an embodiment of the transplantation device that includes a frame with dialysis tubing and without support posts.

FIG. 10 illustrates the scale of embodiments of the disclosure in relation to a 1 mL syringe.

FIGS. 11A-F illustrate the results of a rodent study using an embodiment of the disclosure.

FIG. 12 illustrates dye particles in an alginate bead.

FIGS. 13A-B illustrate in vitro results using an embodiment of an transplantation device.

FIGS. 14A-B illustrate in vivo results using an embodiment of an transplantation device.

FIG. 15 illustrates the presence of arterioles in embodiments of the device.

FIG. 16 illustrates a graph of lifetime decay for calibration to oxygen concentrations.

FIG. 17 illustrates pO₂ measurements upon excitation of an alginate bead.

### DETAILED DESCRIPTION

Disclosed herein are embodiments of a device that can be used for the transplantation of cells and/or tissues. Specifically, embodiments of the disclosed device can establish perfused vasculature in the region of transplantation to provide the necessary nutrients and means of waste removal for the cells/tissue to survive and, in some embodiments, control diabetes. Advantageously, the disclosed device can lead to increased viability in implanted cells.

In some embodiments, Islets of Langerhans, also known as islets, can be incorporated into the device for transplantation of the device. In this specification, “Islets of Langerhans” and “islets” are used interchangeably.

In some embodiment, the transplantation device can house stem cells or other cells derived from stem cells into the transplantation device. In some embodiments, the cells may be insulin secreting cells, however, the cells are not limited to insulin secreting cells. In some embodiments, the transplantation device can house any other type of cell. The type of cell housed within the transplantation device is not limiting.

In some embodiments, the device may be used to direct stem cell differentiation in vivo.

Further disclosed herein is a two phase approach to islet transplantation in which an embodiment of the disclosed transplantation device can be perfused by the host vasculature prior to introduction of islets to the patient. In phase one of an embodiment, embodiments of the device can be implanted in a patients tissue, such as subcutaneous tissue, where the patient's vasculature can invade extracellular matrix (ECM)-containing slits cut through the device, further described in detail below, thereby establishing a microcirculation that passes through the thin dimension of the sheet. The transplantation device may not only be implanted subcutaneously but can also be implanted in other regions of the body of the animal or patient such as, but not limited to, the greater omentum. The location of the implant is not limiting. In phase two, cells can be implanted into the vascularized device.

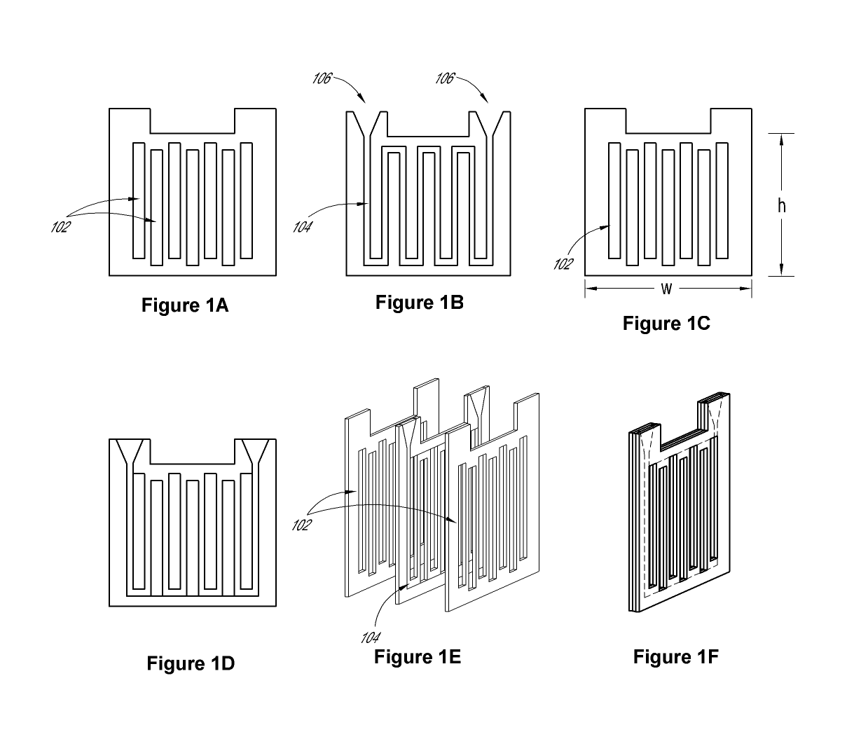

Generally, embodiments of the transplantation device can be formed of a biocompatible material. The device can include slits passing through the device, thus allowing for vascularization of the device, and a fluidic channel within the device. In some embodiments, the fluidic channel may be generally perpendicular to the vascularization direction, although the alignment of the channel and the vascularization direction is not particularly limiting. The fluidic channel can be physically separated from, but in diffusion communication with, the slits in the device. Therefore, upon vascularization of the slits, cells can be inserted into the fluidic channel (e.g., in phase II), such that gas, nutrients and waste can be passed between the cells in the channel and the blood perfusing the slits, and insulin secreted by the islet cells can enter systemic circulation. Embodiments of the device are described below with respect to FIGS. 1A-F. However, this specific configuration is not limiting, and further configurations are described in detail below, along with more detailed description on the configuration shown in FIGS. 1A-F.

FIGS. 1A-F illustrate an example embodiment of a transplantation device. In some embodiments, the transplantation device can be generally thin and flexible, although the specific structural characteristics of the device is not limiting. For example, embodiments of the device can be generally sized and shaped like a business card. In some embodiments, the device can be about 0.1, 0.3, 0.5, 0.7, 0.9, 1, 1.5, 2, 2.5, or 3 inches in length and/or width. In some embodiments, the device can have a thickness of about 100, 200, 300, 400, 500, 600, 700, 800, 900, or 1000 μm. In some embodiments, the device can be square shaped. In some embodiments, the device can be rectangular, circular, or triangular, and the shape of the device is not limiting. The dimensions above are not limiting, and embodiments of the device can be scaled in any dimension.

In some embodiments, the device can be made of biocompatible material, such as alginate, for example scaffold-reinforced alginate, polyethylene glycol (PEG), polydimethylacrylamide (PDMA), polydimethylsiloxane (PDMS), polyacrylonitrile (PAN) or polymethylmethacrylate (PMMA), though the type of material is not limiting to the disclosure. In some embodiments, the device can generally be a structurally solid material. In some embodiments, the device can be a hydrogel.

In some embodiments, vascular endothelial growth factor (VEGF), fibroblast growth factors (FGF) and other growth factors such as, but not limited to, angiopoietins (Ang1 and Ang2), transforming growth factor beta (TGFβ), platelet-derived growth factor (PDGF), hepatocyte growth factor (HGF) can be added into the transplantation device. A cocktail of growth factors can also be used such as, but not limited to, a cocktail composed of VEGF, FGF2, HGF, erythropoietin (EPO), interleukin-6 (IL6), a cocktail composed of FGF-1, FGF-2, VEGF, and TGFβ, or a cocktail composed of VEGF, human growth factor (HGF), TGFα, TGFβ, and heparin. All of the following growth factors or their combinations may be used to promote vascularization. The individual growth factors and/or the angiogenic cocktail of growth factors may be coated on the surface of the device scaffold, or embedded within the material.

As shown in FIGS. 1A-F, in some embodiments the device can contain at least one, preferably a plurality, of slits 102 through the device, which can be used to increase vascular perfusion. In some embodiments, the slits can partially or fully pass through the device. The slits 102 can be any cuts through the device, including, but not limited to, holes, openings, cuts, perforations, etc. The geometry of the slits or vascular openings is not particularly limiting and the slits can take any shape of form consistent with their function of promoting vascularization and enhancing perfusion.

The slits 102 can have a diameter of about 100, 200, 300, 400, 500, 600, 700, 800, 900, 1000, 1100, 1200, 1300, 1400, 1500, 1600, 1700, 1800, 1900, or 2000 μm. In some embodiments, thinner slits, for example between about 200 to 500 μm, can be more vascularized than larger slits. This may occur because the larger slits are more difficult for blood to fill up and remain long enough to form a clot that completely fills the slit. However, with thinner slits, this process is much easier via capillary action, which can keep the blood within the slits and can eventually form a clot that completely fills the slits. In some embodiments, the slits 102 can have a length of about 0.1, 0.3, 0.5, 0.7, 0.9, 1, 1.5, 2, 2.5, or 3 inches, though the length is not limiting.

In some embodiments, the walls of the slits 102 (e.g. the barrier between the fluidic channel 104 and the slits 106) can be about 10, 20, 30, 40, 50, 60, 70, 80, 90, or 100 micrometers thick, although the thickness is not limiting. In some embodiments, the position of the slits 102 in relation to each other may vary. For example, a second slit may be 5 mm away from a first slit, but 8 mm away from a third slit, though the exact numbers are not limiting.

In one aspect, the described device is configured similar to, but opposite, hollow fiber bioreactors, in which perfusion media is circulated in the hollow fibers (capillaries) and the cells are in the interstices surrounding the hollow fibers (see e.g., US 2002/0197713; incorporated herein in its entirety by reference), whereas in the present disclosure, the cells are introduced into the fluidic channels (hollow fibers) after blood flow has been established surrounding the channels.

Embodiments of the transplantation device can also contain at least one inlet port 106 in fluid connection with a fluidic channel 104. Cells can be injected through the inlet port 106 into fluidic channels 104. In some embodiments, the inlet port 106 can be sealed after injection of the cells. In some embodiments, the channels 104 can run along the plane of the surface adjacent to the slits 102. In some embodiments, the fluidic channel 104 can be serpentine. However, the geometry of the fluidic channel 104 is not limiting. In some embodiments, the slits 102 can be vascularized in vivo prior to injection of cells into the channels 104. The channels 104 can have a diameter of about 100, 200, 300, 400, 500, 600, 700, 800, 900, or 1000 μm, and the size of the channels 104 is not limiting.

In some embodiments, different types and configurations of cells can be inserted into the fluidic channels 104. In some embodiments, intact tissue may be used. In some embodiments, the tissue may be disrupted, suspended, homogenized, chopped, etc. In other embodiments, cell suspension may be prepared from the tissue by using standard cell isolation techniques, e.g., collagenase digestion. However, the methodology for forming the tissue is not limiting. In some embodiments, immunoisolation can be achieved by polymerizing alginate, in situ around the cells, thus creating a vascularized sheet of alginate imbedded with cells.

In some embodiments, the fluidic channels can be made of any materials known in the art which are semipermeable, allowing gas, nutrient and waste exchange (and insulin secretion), while retaining the cells and/or tissues. Non-limiting examples of semipermeable materials include one or more of polyacrylonitrile, polyvinylidene fluoride, regenerated cellulose, polysulfone, modified polysulfone, polyamide, cellulose acetate, acrylic copolymer, and cellulose derivatives.

In some embodiments, as the device can be generally thin and planar, multiple layers can be built up like a stack of cards to create a thicker tissue capable of housing a greater number of cells, if needed. Since the axis of the vasculature can be perpendicular to that of the cell containing channels 104, a plurality of devices can be stacked on top of one another without compromising cell perfusion. Ultimately, if islet cells are used, embodiments of this device can improve glycemic control in diabetics by improving islet health and increasing the number of functioning islets after implantation.

In some embodiments, a flat sheet of ultrafiltration, microfiltration, or nanofiltration membranes can be used in the device. These membranes can be polyacrylonitrile (PAN), polyvinylidene fluoride (PVDF) or regenerated cellulose (RC). However, the material is not limiting and other materials can be used. These membranes can be used and modified to house cells, such as islets, and protect the cells from, for example, host immune suppression. The nominal molecular cut-off of the membranes includes, but is not limited to, 1 kilodalton, 5 kilodaltons, 10 kilodaltons, 100 kilodaltons, 200 kilodaltons, 500 kilodaltons, or 1000 kilodaltons. The average pure water flux through the membrane can include, but is not limited to, 350 L/m² h bar. However, other flux, such as 100, 200, 300, 400, 500, 600, 700, 800, 900, or 1000 L/m² h bar can be used. In some embodiments, the membrane can replace the use of alginate. In other embodiments, the membrane can be used with alginate.

In some embodiments, microdialysis tubing can run through the slits in the device, which can help with the vascularization process.

### Preliminary Studies

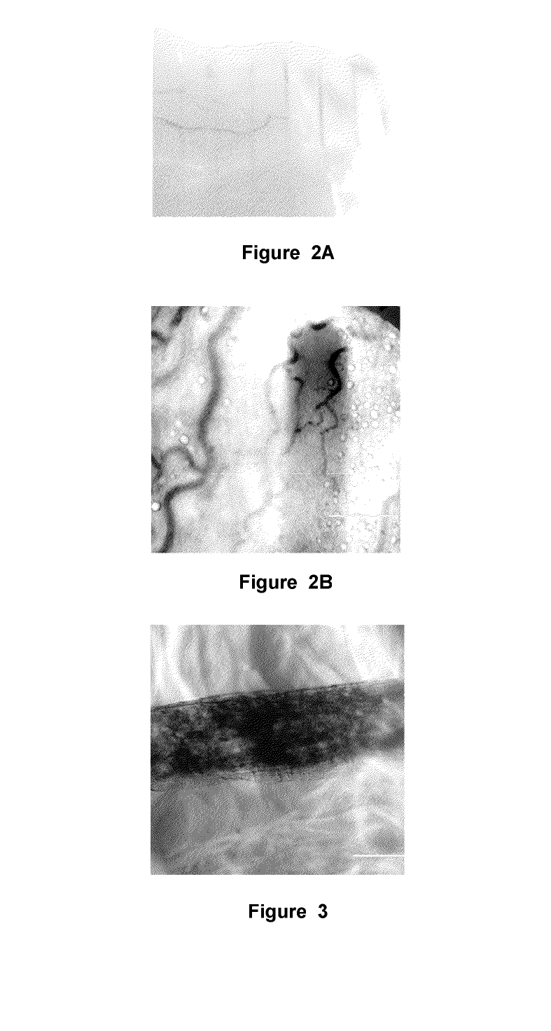

An embodiment of the device's top layer (FIG. 1A) was implanted subcutaneously in a rat for one week. The development of a perfused tissue was observed within the device's slits, indicated by their bright red appearance in FIG. 2A. FIG. 2B illustrates a magnified view of an excised implant, and shows vessels invading a slit. In some embodiments, the device can be constructed from alginate to reduce the fibrotic response. As a proof that alginate can support fluidic channels, a 400 um diameter channel was formed within a 5% alginate gel. A solution of Trypan blue was injected through the channel, which contained the fluid, limited Trypan blue diffusion and allowed for flow (FIG. 3).

### Design Methodology

A housing community is somewhat like a physiological organ. What makes a community? Certainly it is the people functioning within the infrastructure, just as an organ is made of cells functioning within the extracellular matrix (ECM). How is a new community created? It is unthinkable for homeowners to arrive before the housing and infrastructure are established. Yet this strategy captures the current “state of the art” in islet encapsulation.

Long before the arrival of the first homeowner, construction workers establish the infrastructure required to sustain comfortable living: water, power, sewage, telecommunications and roads. Only after inspection deems a home habitable do the homeowners move in. The concept of moving into a home before the electricity, plumbing and sewage are fully functional is unimaginable.

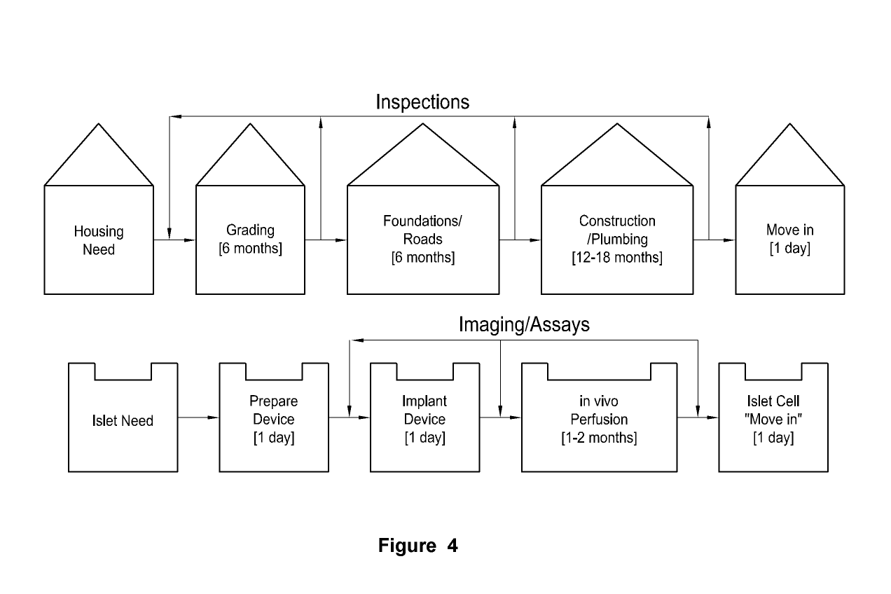

Surprisingly, the modus operandi in islet encapsulation expects the residents, or the islets, to move into their new home while the neighborhood is being plumbed (or vascularized and perfused). In phase one of an embodiment, a business card sized implant (thin sheet) can be implanted in the subcutaneous tissue where the recipient's vasculature invades ECM-containing slits cut through the device, thereby establishing a microcirculation that passes through the thin dimension of the sheet. After a series of “inspections” deem the implant inhabitable for islets, phase one is complete. See FIG. 4. In phase two of an embodiment, islets ‘move in’ to their new home by injection into fluidic channels, which run along the plane of the sheet. The channels are in fluid contact to the vascularized matrix such that each islet can be adjacent to the circulation allowing the normal transport of nutrients (e.g., oxygen and glucose) and waste by diffusion. Immunoisolation can be achieved by polymerizing alginate, in situ around the islets. Additionally, since the device can be thin and planar, multiple layers can be built up like a stack of cards to create a thicker tissue. Since the axis of the vasculature can be perpendicular to that of the islet containing channels, the devices may be stacked without compromising islet perfusion.

### Methodology of Two Phase Approach

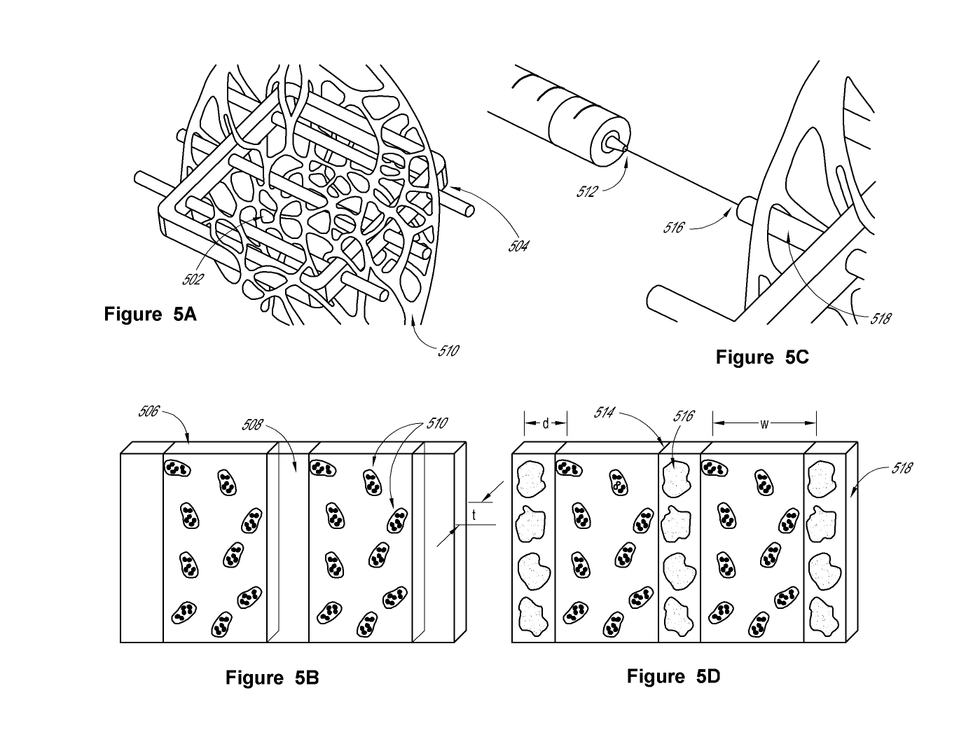

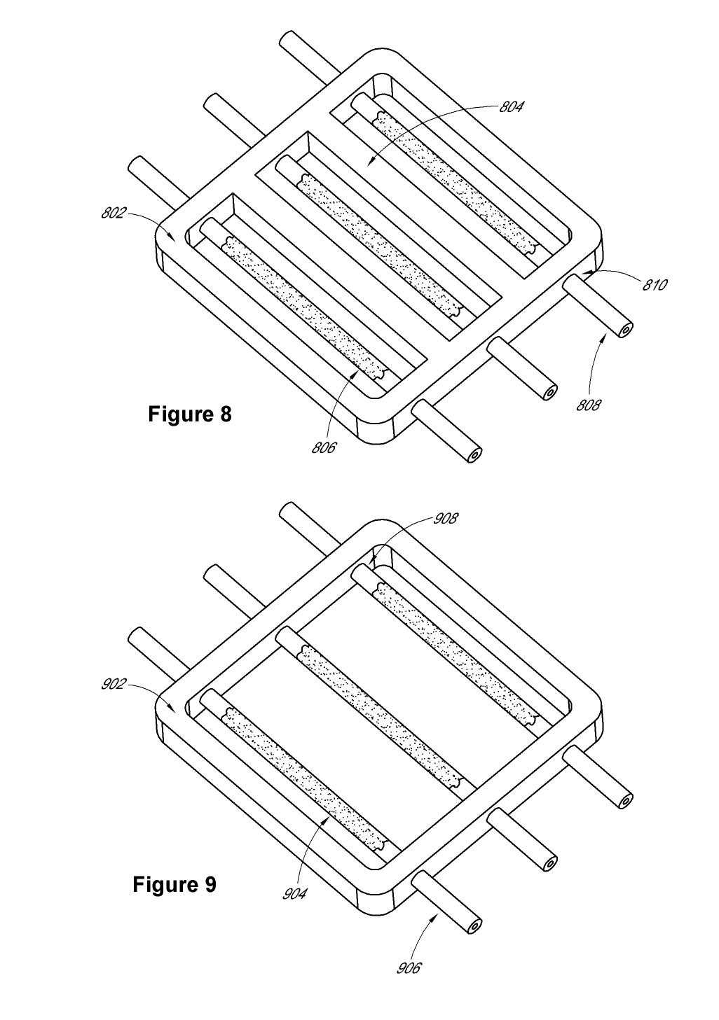

FIGS. 5A-D illustrate a generalized view of an embodiment of a transplantation device. Phase I is shown in FIGS. 5A-B, with a thin sheet comprising a polymer frame 504 with parallel cellulose dialysis fluidic channels 508, such as from FIG. 9, can be implanted into the host, wherein the frame 504 can contain slits 502 between the fluidic channels 508. The fluidic channels 508 can house cells, such as, for example, islets. Upon implantation, the slits 502 can fill with clotted fibrin 506 into which the host vasculature 510 can invade by passing through the dimension t. In the first phase, the device can be implanted and the slits 502 can be perfused with vasculature 510, and thus blood flow, over time.

Phase II is shown in FIGS. 5C-D, which can begin once the device is perfused. The fluidic channels 508 can be filled with islets 516 suspended in alginate 518 or appropriate material. In some embodiments, an injection needle 512 containing the islets 516 can be used, though the method of injection is not limiting. The fluidic channels 508 can run closely along the slits 502, to allow for adequate diffusion of nutrients and removal of waste through porous membrane 514. The islets may not be delivered until the ECM is vascularized and perfused by the host. This can improve glycemic control by improving islet health and increasing the number of functional islets post implanting, thus effectively reversing type 1 diabetes.

### Method of Manufacturing Device

Numerous methods can be used for manufacturing embodiments of the transplantation device. While two such methods are discussed below, other methods can be used as well, and the method of manufacturing is not limiting. For example, portions of the transplantation device can be formed by 3D printing. Further, either method used below, as well as all other potential methods, can be used to form any of the device configurations discussed in detail below.

Sandwich Method

In some embodiments, the device can be manufactured by bonding multiple layers of materials together, such as the embodiment shown in FIGS. 1A-F. In some embodiments, the top and bottom layers are identical to one another. In some embodiments, the top and bottom layers can contain a series of aligned slits 102, whereas a middle layer can include a fluidic channel 106. In some embodiments, the fluidic channel 106 can flare into a triangular flange, or port, 106 at its two ends. The three layers can be bonded to fully enclose the fluidic channel 104, save the ports 106, which allow the fluidic channel 104 to be connected to a soft transdermal injection port such as the i-port (for large animal and clinical studies, Patton Medical Devices, TX). The device may be bonded with medical grade adhesives such as, but not limited to, biocompatible epoxies, UV-cured adhesives, cyanoacrylates, silicone, BioGlue which may consist of fibrin or acrylate, sonic bonding plastics. The device can be preferably bonded with medical grade adhesives that the Class VI criteria set forth by United States Pharmacopeia (USP). The type of bonding is not limiting. Once bonded together, the channel 104 in the middle layer can be completely covered except for inlet holes 106.

In some embodiments, the layers can be laser cut or microfabricated into their final structure, though the type of cutting is not limiting.

In some embodiments, the material of choice for the middle layer can be alginate, which can be directly patterned by laser ablation, or alternatively, using soft lithography, molded from laser cut relief structures into the shapes of FIG. 1. In some embodiments, the middle layer is a different material than the other layers. In some embodiments, all layers are formed from the same material.

Mold Method

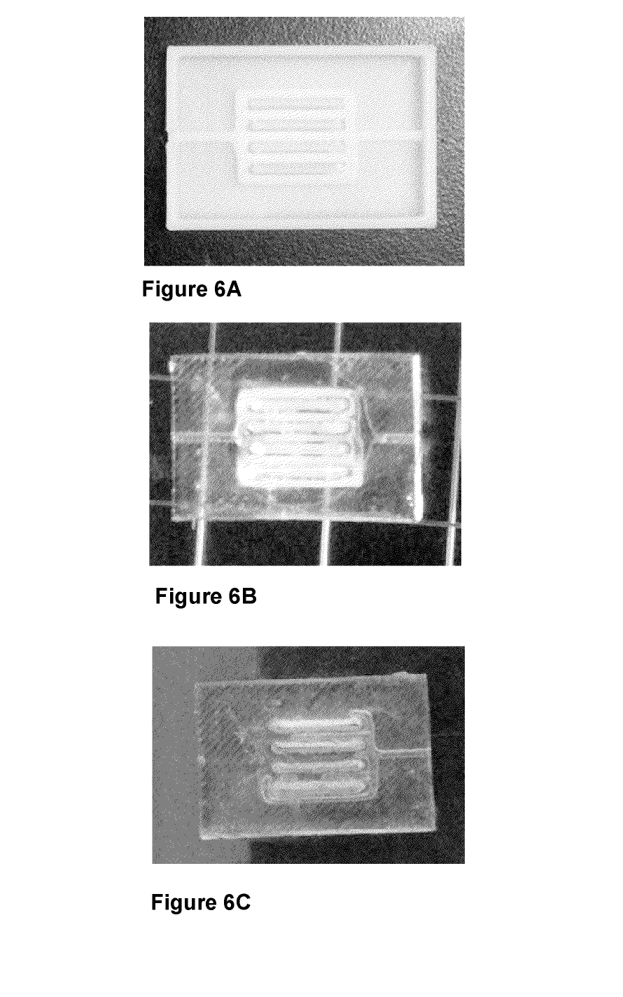

FIGS. 6A-C illustrate an embodiment of a mold that can be used to manufacture a transplantation device. In some embodiments, the mold may be formed through the use of 3D printing, though other methods can be used as well and the method is not limiting. In some embodiments, the mold can be dissolvable. FIG. 6A shows an embodiment of a dissolvable mold. The mold can be configured to receive materials to form a transplantation device, and the type of materials used within the mold is not limiting. For example, (hydroxyethyl)methacrylate (HEMA), polyethylene glycol (PEG), alginate, polylysine, agarose, acrylate copolymers, polydimethysiloxane (PDMS), polymethacrylic acid, 2-methacryloyloxyethyl phosphorylcholine, cellulose sulfate, polyvinyl alcohol (PVA), chitosan, polyarylamide, polysulfone, polyurethane, chondroitin sulfate, polyacrilonitrile, polyacrylonitrile-sodium methallylsulphonate, collagen, fibrin, hyaluronic acid, and any combinations of the listed materials can be used.

Once the material is added into the dissolvable mold, the material can then be polymerized to form the transplantation device. For example, the polymerization can occur through time, heat, or UV exposure, though the type of polymerization is not limiting.

Upon the polymerization of the material, the mold can then be dissolved. For example, the mold can be placed into a bath of solution to dissolve the mold. In some embodiments, citrus oil can be used to dissolve the mold. FIG. 6B shows the mold being dissolved around a device. Upon the mold completely dissolving, the device can remain, as shown in FIG. 6C. In some embodiments, the mold can be removed from the device without dissolving.

### Device Configurations

Disclosed below are configurations for embodiments of an transplantation device. Each of the configurations can be formed by the above described methods, though other methods could be used as well.

In some embodiments, as shown in FIGS. 1A-F, the device can be made of a plurality of different layers. FIG. 1A shows an embodiment of a bottom layer, which can contain slits 102 for vascularization. FIG. 1B shows an embodiment of a middle layer which can contain fluidic channels 104 for containing cells, such as islets. FIG. 1C shows an embodiment of a top layer, which can be similar in shape to the bottom layer, though this is not necessary. These layers can be sandwiched together to form a final transplantation device, as shown in FIGS. 1D-F. Once sandwiched together, the channel 104 in the middle layer can be completely covered except for inlet holes 106.

As shown in FIGS. 1A-F, in some embodiments the device can contain at least one, preferably a plurality, of slits 102 through the device. The slits 102 can be any cuts through the device, including, but not limited to, holes, openings, cuts, perforations, etc. In some embodiments, the slits 102 can be cut out through a second round of laser cutting, though the method of manufacturing is not limiting.

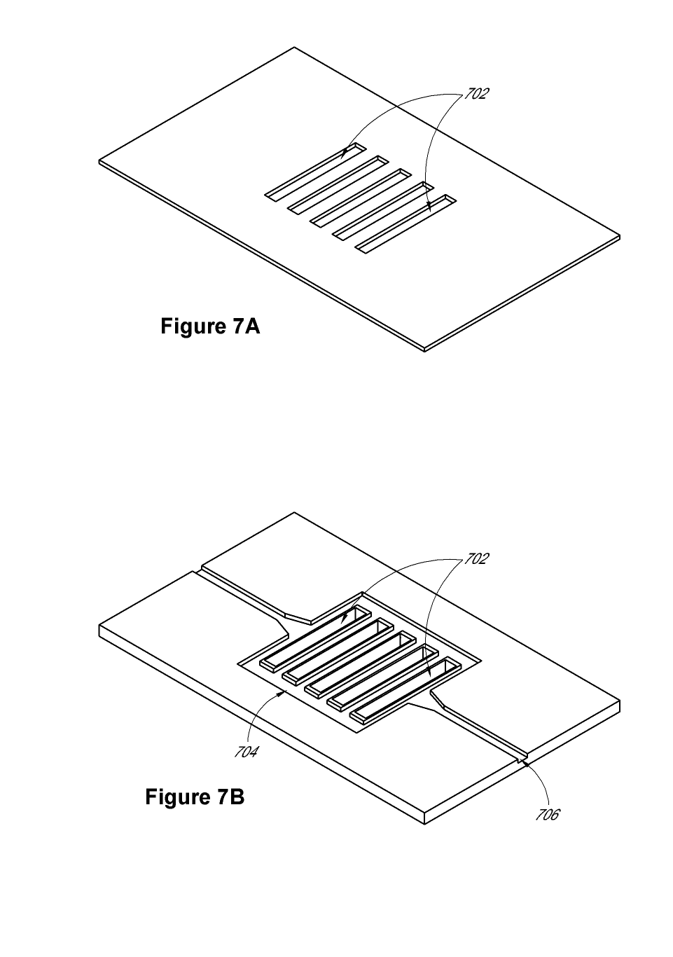

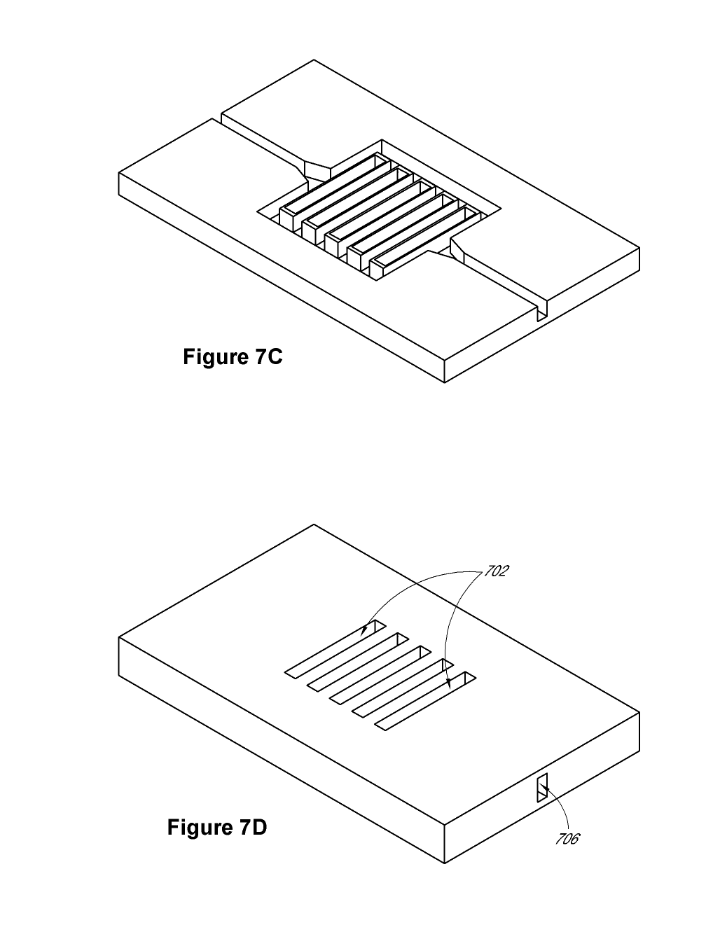

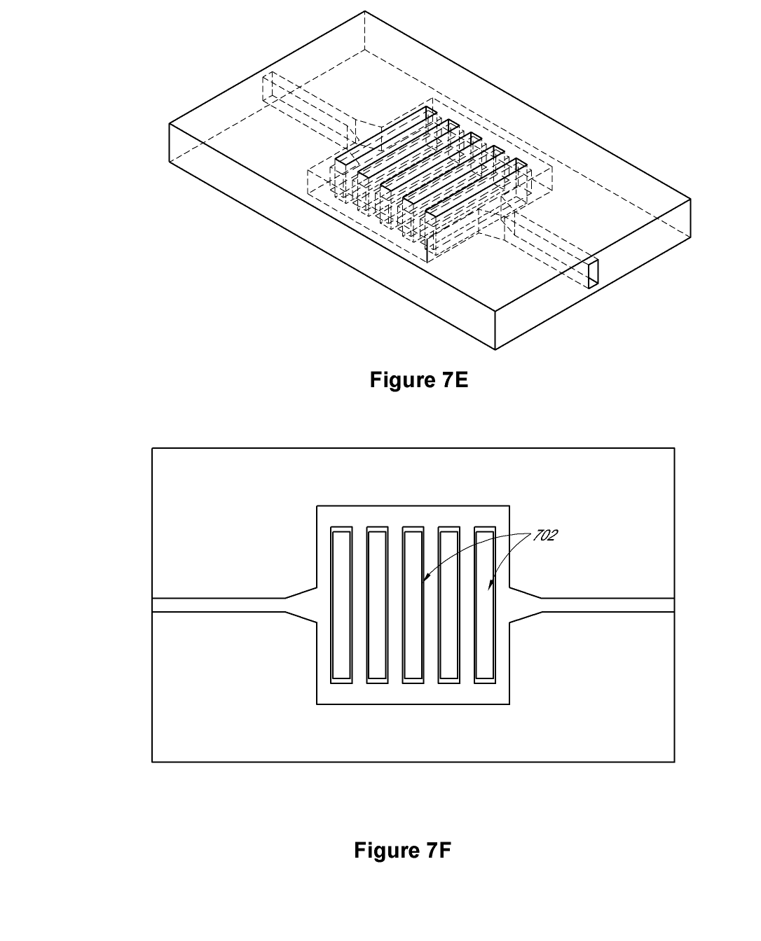

Further, FIGS. 7A-F illustrate an embodiment of an transplantation device. FIG. 7A illustrates a top layer of the device having a series of slits 702 passing through the device. As shown, the device has four slits 702, though the number of slits is not limiting. The device could have, for example, 1, 2, 3, 4, 5, 6, 7, 8, 9, or 10 slits 702. As mentioned above, the slits 702 can have different thicknesses, though smaller thickness can be advantageous in increasing vascularization.

FIG. 7B illustrates the inside of an transplantation device. As shown, the slits 702 pass completely through the device. Further, the device can contain a fluidic channel 704, which can be used to contain cells. The fluidic channel 704 can pass around each of the slits 702. Accordingly, nutrients and waste can diffuse through the device between the channel 704 and slits 702. In some embodiments, the fluidic channel 704 can also include an inlet 706 for insertion of cells into the channel 704. In some embodiments, more than one inlet 706 can be used.

FIG. 7C illustrates the inside of an embodiment of an transplantation device, similar to the device shown in FIG. 7B. However, FIG. 7C shows a greater height to the channel 704 and slits 702.

FIG. 7D illustrates a fully constructed embodiment of an transplantation device. As shown, the slits 702 pass through the device. Further, the only direct access to the fluidic channel 704 is through the inlet 706. FIG. 7E illustrates the internal structure of the fully constructed embodiment of FIG. 7D. FIG. 7F illustrates a top-down viewpoint of FIG. 7E. As shown, the slits 702 can pass completely through the device.

Another embodiment of the transplantation device is shown in FIG. 8. The transplantation device can be made of a frame 802 with one or more posts 804 traversing the device for support. The frame 802 has a number of holes 810 on opposite sides of the frame to allow dialysis tubing 808 to be pulled through the frame 802 wherein the dialysis tubes 808 are parallel to the posts 804. The tubes 808 can act as the fluidic channel. The dialysis tubes 808 can contain particles 806 in channel which can be, for example, islet cells. The dialysis tubes 808 may have a diameter of between about 0.1-100 mm, 1-100 mm, 10-100 mm, 10-50 mm, or 0.1-10 mm. The dialysis tubes 808 can be made of, but not limited to, regenerated cellulose. In some embodiments, the device can contain a plurality of tubes 808. In some embodiments, the device can contain one longer tube 808, throughout the frame, such as a serpentine tube. The frame 802 is not be limited to a square shape but may take on other shapes such as circle, rectangle, etc. The dialysis tubes are connected directly or indirectly to a soft transdermal injection port, such as the i-port. In some embodiments, the frame 802 can further contain a top and bottom portion covering the tubing 808, thereby lending more structural support. The top and bottom portion can contain slits, such as those described above, so that the sides of the tubes 808 can still be exposed to vascularization. In some embodiments, the top and bottom portions can contain short side walls to at least partially retain the tubes 808, providing for further structural support.

Another embodiment of the transplantation device is shown in FIG. 9. The transplantation device can be made of a frame 902 with no posts traversing the device for support, dissimilar from the embodiment shown in FIG. 8. The frame 902 can have a number of holes 908 on opposite sides of the frame 902 to allow dialysis tubing 906 to be pulled through the frame 902. The tubes 906 can act as the fluidic channel. In some embodiments, the device can contain a plurality of tubes 906. In some embodiments, the device can contain one longer tube 906, throughout the frame, such as a serpentine tube. The dialysis tubes 906 can contain particles 904 in channel which can be, for example, islet cells. The dialysis tubes 906 may have a diameter of between about 0.1-100 mm, 1-100 mm, 10-100 mm, 10-50 mm, or 0.1-10 mm. In some embodiments, the frame 902 can further contain a top and bottom portion covering the tubing 906, thereby lending more structural support. The top and bottom portion can contain slits, such as those described above, so that the sides of the tubes 906 can still be exposed to vascularization. In some embodiments, the top and bottom portions can contain short side walls to at least partially retain the tubes 906, providing for further structural support.

When using dialysis tubing, such as in the embodiments shown in FIG. 8 and FIG. 9, initially prior to implant, the dialysis tubing of the transplantation device can be filled with saline or unpolymerized alginate so that bacteria may not invade and contaminate the device. The entire device may then be sterilized by conventional methods such as sterilization with ethanol or irradiating the transplantation device with UV radiation.

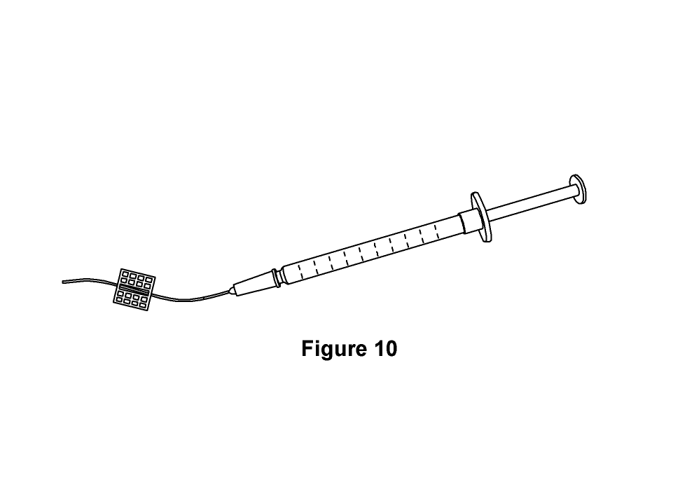

FIG. 10 illustrates a general scale of embodiments of the disclosed device in any of the above configurations with respect to a 1 mL syringe.

### Validation of In Situ Alginate Polymerization and Transport Characteristics

Using the injection ports, non-polymerized alginate containing 100 um diameter polystyrene microbeads (as surrogates for islets) can fill the fluidic channel and can be cross-linked in situ by immersing the device in phosphate-buffered saline (PBS) supplemented with Ca++. By microscopy, it can be determined if the beads are evenly distributed within the channel. A fluorescent molecule such as Alexa-488, or Alexa-488-Dextran of low molecular weight can be then added to the PBS. The fluidic channels can be imaged serially by laser scanning confocal microscopy to determine the transport rate of the molecules between the fluidic channels and the slits/exterior of the device. Transport rates can be calculated from models of diffusion fit to the increase of fluorescence within the channel over time.

### Device Vascularization and Perfusion

Embodiments of the devices were implanted into both Nude and immunocompetent inbred Balb/c diabetic mice. Diabetes was induced by intraperitoneal injection of 180 mg/kg Streptozotocin (STZ), and confirmed by three consecutive days of hyperglycemia (>350 mg/dl glucose) as measured using tail vein blood. To promote vascularization, slits were filled with (a) autologous blood (occurs naturally during implantation), (b) fibrin purified from donor mice, or (c) both fibrin and endothelial progenitor cell (EPC) derived endothelial cells from donor animals.

In vivo, the animal or patient should have sufficient growth factors to promote vascularization. Therefore the device may be fabricated to not include growth factors and to rely on the growth factors that are found naturally in the animal or the patient. However, the rate of vascularization can be slower than that of a device with growth factors incorporated into the device or later added into the device.

In some embodiments, the device may include the incorporation of homologous cells such as, but not limited to, blood cells or endothelial progenitor or colony forming cells from cord or peripheral blood, or marrow-derived cells. These homologous cells would produce the growth factors to promote or accelerate vascularization of the device.

Devices were explanted at weeks 1, 2, and 4 and sectioned for histology. Samples were paraffin embedded and sectioned for histology. Sections were stained for CD31, specific for endothelial cells, and counter stained with H&E to determine the percentage of new vessels that are perfused within the slits of the device. Additionally, new ECM was detected by staining for collagen and elastin and imaging collagen by second harmonic generation microscopy and elastin by two-photon auto fluorescence. Periodically the progression of vascularization and perfusion was monitored non-invasively by biophotonic techniques and by analytical measurements of glucose levels within the device. Specifically, non-invasive monitoring by multiphoton microscopy and laser speckle imaging provided measures of perfusion within the device. Prior to surgical excision of devices, the fluidic port was flushed with PBS, being careful to flush the exact volume of the fluidic channel and to recollect the fluid. The procedure was repeated after ten minutes, half hour and one-hour durations. The concentration of glucose in the collected fluid was measured and compared to blood glucose levels.

### Islet Preparation

Islets were isolated from the pancreas using methods of intraductal delivery of enzyme (collagenase) into the pancreatic duct. The distended pancreas was then mechanically and enzymatically dissociated before purification of the islets from the exocrine tissue by differences in their density. Islets were collected and washed in tissue culture media supplemented with serum and supplements.

The final criteria for islet product release included an islet infusion compatible with the ABO blood group, an islet mass of 5000 islet equivalents per kilogram or more (on the basis of the weight of the recipient), an islet purity of 30% or more, a membrane-integrity viability of 70% or more, a packed-tissue volume of less than 10 ml, negative Gram's staining, and an endotoxin content of 5 endotoxin units per kilogram or less (on the basis of the weight of the recipient).

Islets were prepared locally in Good Manufacturing Practice-grade facilities at each of the nine sites, according to identical standard operating procedures. The pancreas from a donor was distended by controlled ductal perfusion with the use of common batch lots of Liberase human islet enzyme (Roche Diagnostics), previously validated at the participating sites. The pancreas was digested in a Ricordi chamber and purified on continuous Ficoll gradients on a cooled apheresis system (model 2991, Cobe Laboratories). The islets were then washed and resuspended in transplant medium (Mediatech).

### Islet Viability and Glycemic Control

A set of diabetic Nude mice received one implant per mouse. Once the device was perfused and ready to accept islets a mixture of non-polymerized alginate and 2000 islets were perfused into the fluidic channel. The alginate polymerized in situ by the diffusion of interstitial calcium ions. Calcium ions were injected into to the implant to polymerize the alginate, or calcium naturally occurring in the animal or patient may polymerize the alginate. Blood glucose was measured at intervals, e.g. from about 1-14 times per week, more preferably about 3 times per week until euglycemia is observed for about 30-60 consecutive days. The device was then removed and histology can be performed. Mice were housed until a return to hyperglycemia is observed, and sacrificed for histology evaluation. Immediately after device extraction, islets were stained with Dithizone to test for insulin production and Syto/EB to measure islet cell viability.

A set of pigs also received the implant through a similar procedure as above. The pigs were made diabetic with the beta cell toxin, streptozocin at dose of 150 mg/kg. The pigs then received the implantable device. Pig blood glucose levels were monitored via a cannula placed in a vein in the ear.

### Islet Dose Escalation

To determine the number of islets required to induce and sustain insulin independence after implantation, the method was repeated using different numbers of islets per mouse, for example about 500, 1000 or 2000 islets per mouse transplant.

### Rodent Studies

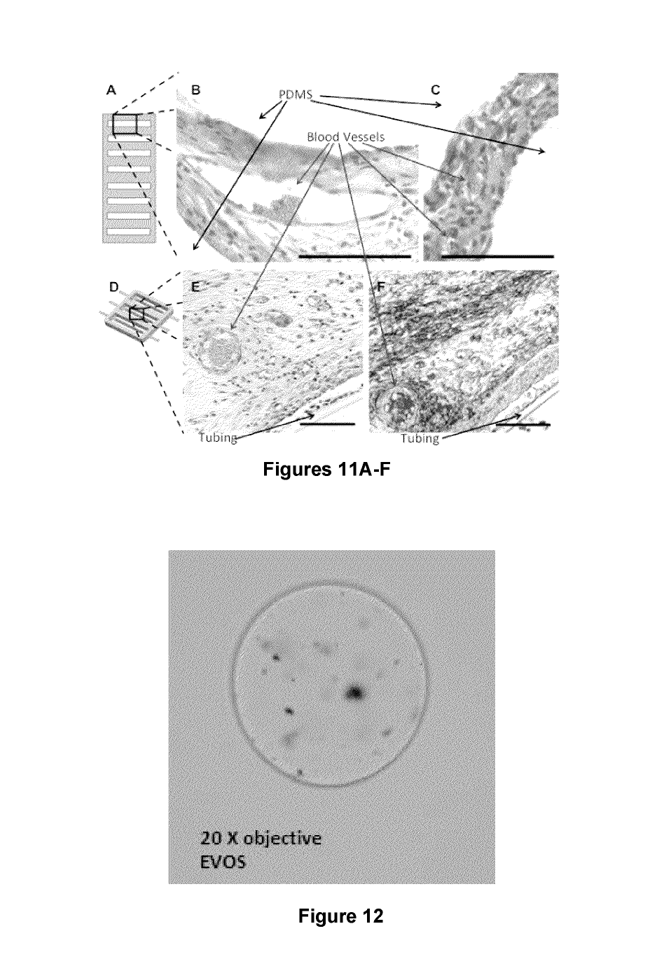

Pre-vascularization and perfusion in vivo of the device described above was performed in a rodent. PDMS and PMMA sheets were implanted subcutaneously within Sprague-Dawley rats for about two weeks. The sheets contained laser-cut slits ranging from about 200 μm to about 1 mm in width, such as those shown in FIG. 11A. After one week, fibrosis was observed along the edge of the sheet, as shown in FIG. 2A. However, the faces of the sheet remained relatively transparent. A 10× magnified view shows bright red vessels infiltrating a slit, as shown in FIG. 2B. The slit appears dark due to multiple light scattering within the newly formed tissue. Histological sections of slits not containing microdialysis tubing were stained with Hematoxylin and eosin (H&E). As shown in FIGS. 11B-C, imaging confirms the formation of “large” and “small” microvessels within the slits. Perfused arterioles and venules were easily identified by erythrocyte-containing lumens. Perfused vessels are marked by the red blood cells they carry.

Next slits containing microdialysis tubing were implanted. In some embodiments, the microdialysis tubing can run in the slit along the long axis of the device, as shown in FIG. 11D. In some embodiments, new vessels can form in the perpendicular direction across the thin dimension of the device. As shown in FIGS. 11E-F, perfused vessels can also be seen in close proximity to the tubing in H&E stains and CD31 staining. As shown in FIG. 11F with the red overlay, CD31 (Mouse Anti-Rat PECAM-1, Millipore) staining specific to vascular endothelial cells confirms the development of a dense capillary network within about 50 μm of the tubing surface, and showing perfused vessels within 100 μm of the tubing wall (e.g., arrows on lower right of FIG. 11E and FIG. 11F. Therefore, in some embodiments, the device can have sufficient perfusion to support pancreatic islets within the tubing in vivo. For the above figures, the scale bar is 100 μm.

An in vitro study was performed to confirm that islets remain viable within the tubing and can deliver insulin through the tubing wall. Porcine Islets were cultured within microdialysis tubing for about 8 days, where the tubing was placed within a petri dish and submerged in culture media. As controls, islets isolated from the same pancreas within non-porous polyethylene (PE) tubing or within a Petri dish without tubing were cultured. After the 8 days islets viability was assessed by a standard live/dead assay comprising propidium iodide (PI, “Dead”) and Newport Green (NG, “alive”). As shown in Table 1, islets cultured within microdialysis tubing showed high viability as compared to both islets cultured without tubing and those cultured with PE tubing.

TABLE 1
Live-dead stain confirms islet viability within microdialysis
tubing: live/(live + dead) × 100%

| Islet Culture Condition | Live Islet % (±STD) |
|---|---|
| Islets Suspended in Culture dish (no tubing) | 43.5 (±32.3) |
| Islets in P-50 Tubing | 38.2 (±20.7) |
| Islets in microdialysis Tubing | 70.7 (±33.8) |

### Vascular Profusion Methodology

In some embodiments, it can be advantageous to determine the amount of vascular profusion of the transplantation device. One such method for determining the vascularization is through the use of oxygen sensitive dyes, where oxygen concentration in or around the implant can be measured. In some embodiments, tissue pO2 measurements can be taken at the implant site at different timepoints after the device has been implanted to see whether the oxygen level around the implant is increasing or decreasing. These measurements around the area of the implant may be useful for determining the amount of vascularization around the device, but these measurements may not tell us what the oxygen level is inside the implant. It can be advantageous to know the oxygen level is inside the implant because eventually there will be cells loaded inside the implant that will need a certain amount of oxygen to survive. Knowing the amount of oxygen in the implant at different timepoints may help determine whether or not the implant itself, or the way it is implanted, is providing sufficient oxygen for cell survival.

There are commercial devices for measuring pO2 levels around the implant (e.g. PeriFlux System 5000) but no commercial system would be able to measure the level of oxygen inside embodiments of the disclosed implant. To make that inner oxygen measurement, disclosed herein is a method to incorporate oxygen sensitive dyes into the implant material so that the oxygen level can be measured from outside the body with light.

In some embodiments, an oxygen sensitive dye, such as, but not limited to, metalloporphyrin can be ground up into small particles (about 1-200 micron in diameter, though the size is not limiting) and mixed into a liquid synthetic or natural material, that can later be polymerized, or otherwise hardened into a gel or solid. The dye can be incorporated into the device through either of the manufacturing methods described above.

In some embodiments, platinum tetraphenyl tetrabenzoporphyrin (PtTPTBP) (Frontier Scientific) can be mixed with polystyrene and dissolved in chloroform. A thin sheet of the dye/polystyrene mixture can be formed by pipetting the liquid mixture onto a glass slide and allowing the solvent (the chloroform) to evaporate. Then a razor can be used to break the thin dye layer up into fine particles. These particles can then be added to a liquid hydroxyethylmethacrylate (HEMA)/polyethyleneglycol (PEG)/water/photoinitiator mixture and can be shaken to disperse the particles evenly throughout the liquid. The liquid/dye particle mix can then be pipetted onto a glass slide and cured under UV light for about 5 minutes.

In some embodiments, the dye can be mixed with alginate or other permeable materials which can form into beads which contain the dye. FIG. 12 illustrates an embodiment of dye particles in an alginate bead. The dye can also be mixed with saline and injected into the channels of the device.

Another way to incorporate the oxygen-sensitive dye into embodiments of the device is to load the particles into the fluidic channels formed in the device. The channels of the device will eventually be loaded with cells, so measuring the oxygen levels within the channels will provide an even more accurate measurement of the oxygen level that the cells will experience. This can be done during the manufacturing of the device, or after the device is fully finished. For example, in some embodiments the dye can be added with the cells into the fluidic channel of the device.

In some embodiments, the dye can emit a fluorescent signal which can be detected by a sensor. The fluorescent lifetime of the dye can be quenched (e.g. lowered) where more oxygen is present, which can allow for determining the level of perfusion of the device. As the patient can inhale gas with different oxygen compositions (e.g., 100% O2, 80% O2, 60% O2), the rate at which the dye reacts to changes in inhaled gas can correlate with the amount the device is perfused. The quicker the reaction of the dye to changes in inhaled gas indicates a well perfused device, as vasculature carries the gas inhaled, and the more vasculature running through the device, the quicker the inhaled gas is carried to the device. Upon determination of the perfusion of the device, it can be determined when the channels are an ideal environment for cells to be housed, and thus cells can be introduced.

For testing purposes, the gel was formed into a simple disk, but the liquid mixture can be formed into a shape by filling a mold with the mix and curing it in the mold. For example, the gel can be formed into one of the embodiments of the above-disclosed transplantation devices.

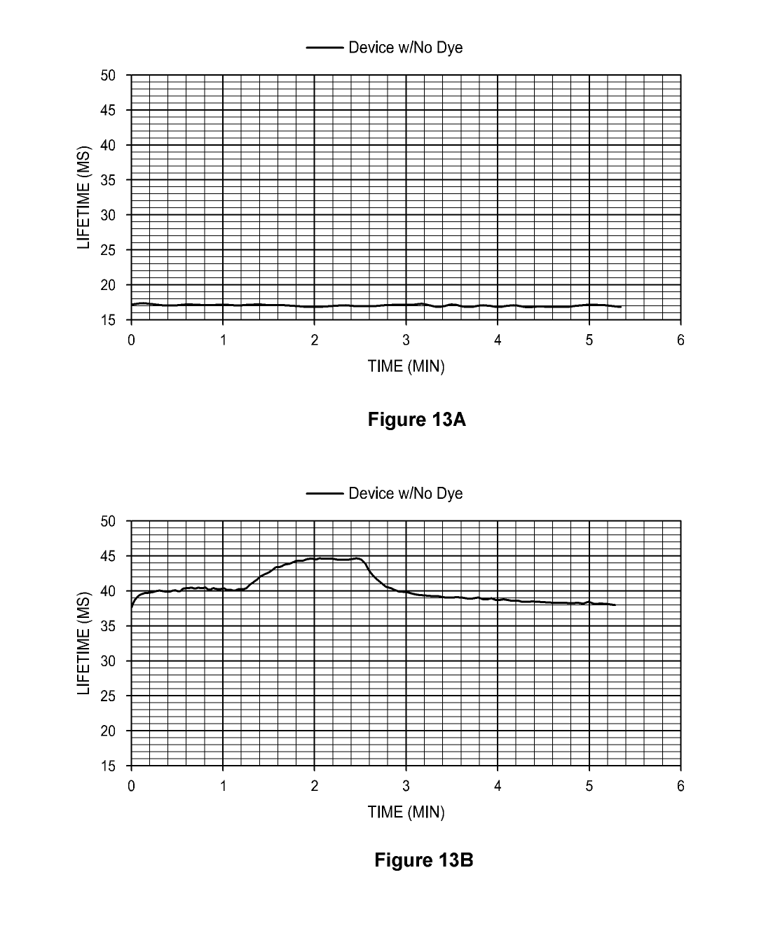

FIGS. 13A-B illustrate the results of the dye injection in vitro. The dye was injected into channels of an embodiment of the device, and the channels were closed off, thereby isolating the dye within the channels. Accordingly, there is no perfusion or fluid flow between the outside and the inside of the device.

The device was then submerged in 1×PBS solution, and gas pumped into the solution. First, room gas was pumped into the solution, then Argon gas, then room gas. Accordingly, the only way for oxygen to enter the channels was through diffusion. Oxygen within the outside solution passes through the hydrogel via diffusion, entering the channels within the device, where the dye within the channels detect oxygen and have their fluorescent lifetime quenched.

FIG. 13A illustrates a control device with no dye in the device. First room are was added into the PBS solution, followed by about 2 minutes of Argon gas, followed by about 3.5 minutes of room air. FIG. 13B illustrates a device with the dye. First room are was added into the PBS solution, followed by about 5 minutes of Argon gas, followed by about 10.5 minutes of room air. As shown, the lifetime fluorescence of the oxygen dye fluctuated based on the oxygen that entered into the system. The changes in oxygen were quickly detected, as the moment the gases were switched, the sensor detected the change in fluorescence lifetime.

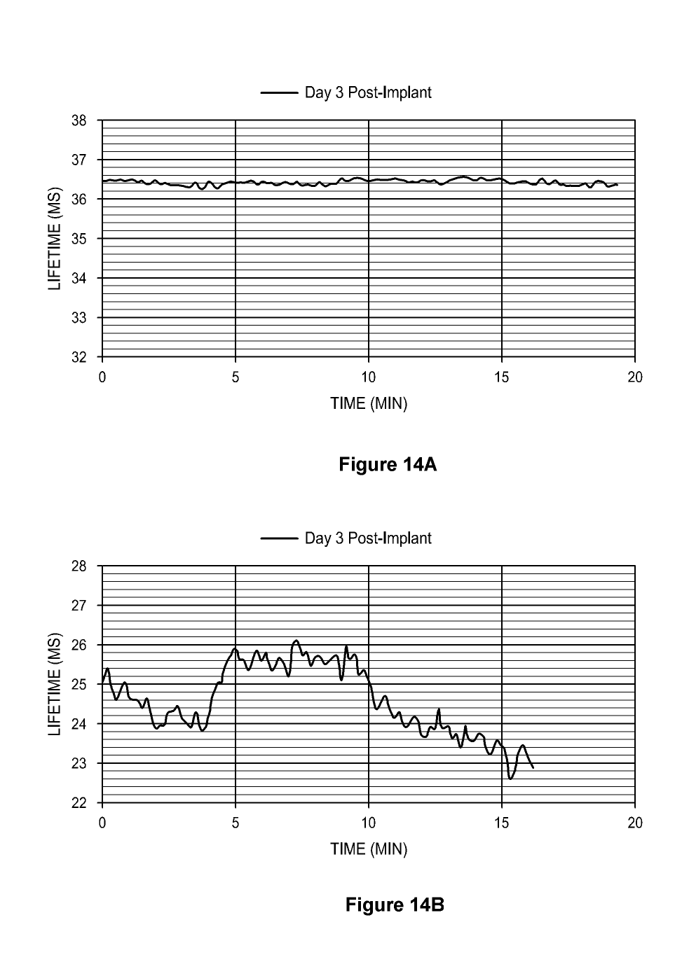

FIGS. 14A-B illustrate in vivo testing using an oxygen dye. The injected dye was mixed with 1×PBS into the channels of the device, and the seals were closed. A small incision was made on the dorsal skin of the Sprague-Dawley (SD) rate, where the device was implanted in the subcutaneous region adjacent the rodent's spine. Once implanted, the incision was stapled or sutured shut.

FIG. 14A illustrates day 3 post implant, with the fluorescent sensor placed on the skin of the rodent above the middle region of the implant. The rodent was breathing 100% oxygen initially, but changed to 60% oxygen at about 5 minutes, then back to 100% oxygen at about 12.5 minutes. As shown in FIG. 14A, there are no dye dynamics detected. The vessels are still far from the implant, and there isn't enough vasculature invading the slit at this point because the wound is still healing. So applying changes to oxygen the rodent is inhaling will not be detected by the dye because there is not enough vasculature in the slits of the device to carry oxygen that the dye will detect.

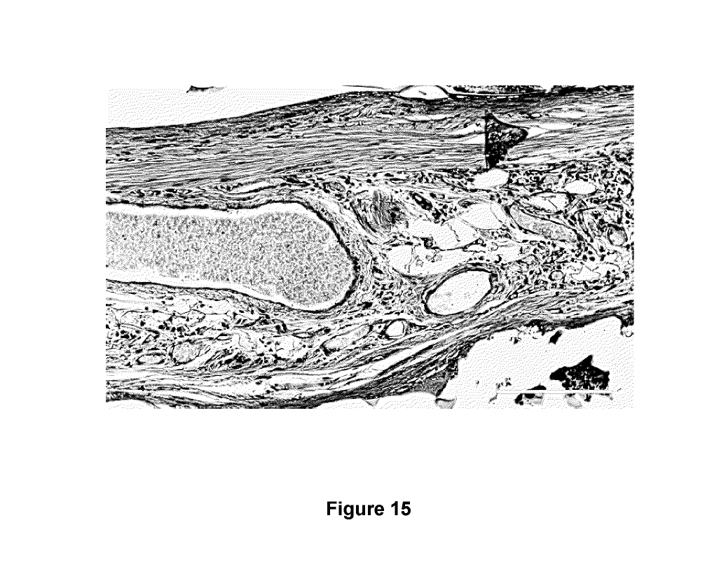

On the other hand FIG. 14B illustrates a day 10 post implant, with the sensor placed in the same position as in day 3. The rodent was initially breathing 100% oxygen, was changed to 60% oxygen at minute 3.5, and back to 100% oxygen at minute 9. As shown in FIG. 14B, a dye dynamic correlating with the time at which a change in oxygen concentration that the rodent was inhaling occurred is detected. This indicates that the slits are vascularized and that the oxygen from this vasculature is diffusing through the walls of our device, into the channels of our device, where they are being detecting by the dye housed within those same channels. A small change in lifetime may indicate arterial blood is near-by. FIG. 15 supports this and shows the presence of arterioles in the slits of the device.

Further testing was performed on the oxygen sensitive dyes. As above, the polymerized dye mixture was crushed into small particles, then the particles were mixed with Phosphate Buffered Saline (PBS) and the mixture was shaken to disperse the particles throughout. Next, the dye/PBS mixture was drawn up into a syringe and injected into the channels of the formed devices. Glue was placed over the ends of the channels after they are loaded to seal the dye/PBS inside. Measurements of pO₂ were then be obtained by probing the particles that now lie in the channels.

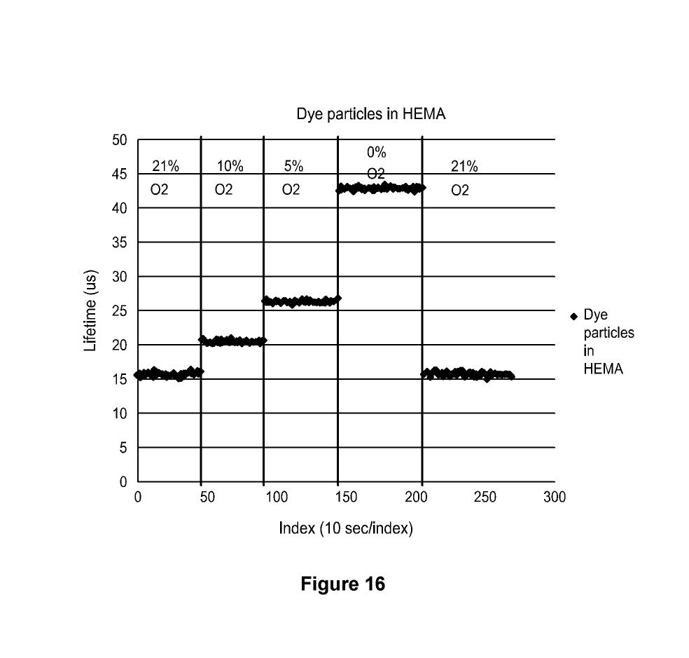

The dye particles in cured HEMA gel were tested in the lab for sensitivity to oxygen between 0-21% O2 (no oxygen to room air quantity of oxygen) with calibrated gas mixtures. By exciting the dye particles with light and measuring their emitted light's lifetime decay a calibration of dye lifetime values to oxygen concentrations could be made, as shown in FIG. 16.

Using a different matrix for the dye particles (alginate) lifetime values can be obtained that can be related to oxygen content from implanted gels loaded with dispersed dye particles in rats in vivo.

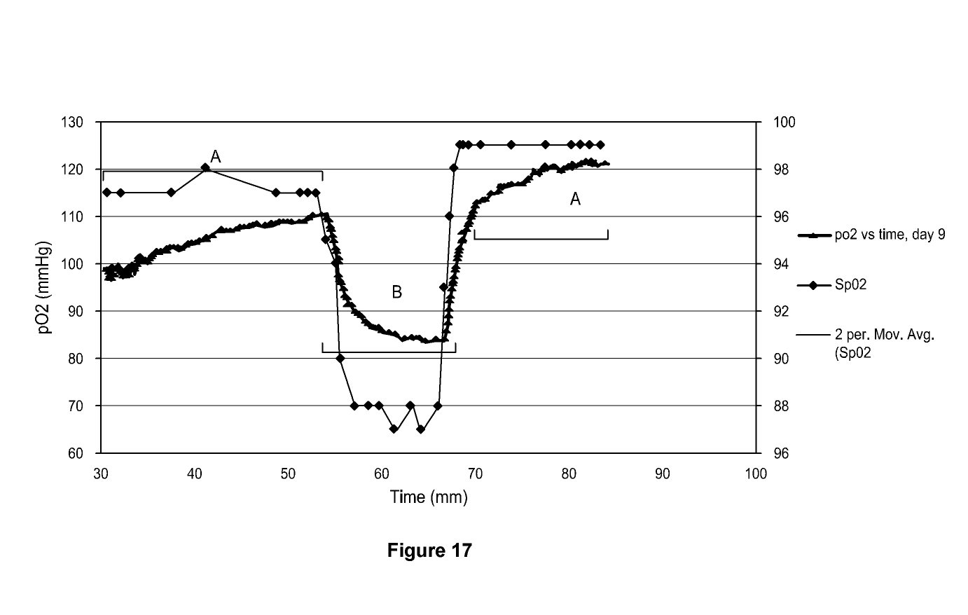

FIG. 17 shows continuous pO2 measurements taken by exciting the alginate beads with dye particles with light through rat skin and collecting the emitted light from the dye in the beads on a detector and calibrating that signal to pO2 levels (red data points). The oxygen measured by the beads correlates with oxygen reported by a pulse-oximeter spO2 (blue data points) when the rat breaths either 100% oxygen or 21% oxygen (room air). There is a delay due to physiologic differences in oxygen transport between arterial blood (pulse-ox spO2) and oxygen in the interstitial space where our sensor beads are implanted (bead pO2).

From the foregoing description, it will be appreciated that inventive devices and approaches for transplantation device have been disclosed. While several components, techniques and aspects have been described with a certain degree of particularity, it is manifest that many changes can be made in the specific designs, constructions and methodology herein above described without departing from the spirit and scope of this disclosure.

Certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as any subcombination or variation of any subcombination.

Moreover, while methods may be depicted in the drawings or described in the specification in a particular order, such methods need not be performed in the particular order shown or in sequential order, and that all methods need not be performed, to achieve desirable results. Other methods that are not depicted or described can be incorporated in the example methods and processes. For example, one or more additional methods can be performed before, after, simultaneously, or between any of the described methods. Further, the methods may be rearranged or reordered in other implementations. Also, the separation of various system components in the implementations described above should not be understood as requiring such separation in all implementations, and it should be understood that the described components and systems can generally be integrated together in a single product or packaged into multiple products. Additionally, other implementations are within the scope of this disclosure.

Conditional language, such as “can,” “could,” “might,” or “may,” unless specifically stated otherwise, or otherwise understood within the context as used, is generally intended to convey that certain embodiments include or do not include, certain features, elements, and/or steps. Thus, such conditional language is not generally intended to imply that features, elements, and/or steps are in any way required for one or more embodiments.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than or equal to 10% of, within less than or equal to 5% of, within less than or equal to 1% of, within less than or equal to 0.1% of, and within less than or equal to 0.01% of the stated amount.

Some embodiments have been described in connection with the accompanying drawings. The figures are drawn to scale, but such scale should not be limiting, since dimensions and proportions other than what are shown are contemplated and are within the scope of the disclosed inventions. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various embodiments can be used in all other embodiments set forth herein. Additionally, it will be recognized that any methods described herein may be practiced using any device suitable for performing the recited steps.

While a number of embodiments and variations thereof have been described in detail, other modifications and methods of using and medical applications for the same will be apparent to those of skill in the art. Accordingly, it should be understood that various applications, modifications, materials, and substitutions can be made of equivalents without departing from the unique and inventive disclosure herein or the scope of the claims.

What is claimed is:

1. A device for cell transplantation comprising:
a biocompatible frame configured to be inserted into tissue;
at least one slit passing through the frame, wherein the at least one slit is sized and configured to allow vascular perfusion through the at least one slit; and
a fluidic channel located within the frame and comprising a semipermeable surface region configured to retain cells while allowing certain dissolved molecules to diffuse between the fluidic channel and the at least one slit.

2. The device of claim 1, further comprising at least one inlet/outlet port in fluid communication with the fluidic channel.

3. The device of claim 2, wherein the at least one inlet/outlet port is configured to be sealed.

4. The device of claim 1, wherein the fluidic channel is configured to retain islet cells.

5. The device of claim 1, wherein the frame is formed from a plurality of layers bonded together.

6. The device of claim 1, wherein the frame is monolithic.

7. The device of claim 1, wherein the frame is a hydrogel.

8. The device of claim 1, wherein the frame is formed from a material selected from the group consisting of alginate, polydimethylacrylamide (PDMA), polydimethylsiloxane (PDMS), polyacrylonitrile (PAN) or polymethylmethacrylate (PMMA).

9. The device of claim 1, further comprising a plurality of slits.

10. The device of claim 1, further comprising a plurality of fluidic channels.

11. The device of claim 1, wherein the semipermeable surface region of the fluidic channel comprises dialysis tubing.

12. The device of claim 1, wherein the biocompatible frame is hollow and at least partially defines the fluidic channel.

13. The device of claim 1, wherein the fluidic channel is serpentine.

14. The device of claim 1, further comprising an oxygen sensitive dye incorporated into the device.

15. The device of claim 14, wherein the oxygen sensitive dye has a fluorescence lifetime based on oxygen levels.

16. The device of claim 1, wherein one or more agents promoting vascularization are incorporated into the device.

17. The device of claim 16, wherein the one or more agents promoting vascularization are autologous blood, fibrin purified from donor mice, VEGF, or other growth factors.

18. A method for making an transplantation device for islet transplantation comprising:
fabricating a bottom layer having at least one slit;
fabricating an inner layer having a fluidic channel comprising a semipermeable surface region and an injection port;
fabricating a top layer having at least one slit, the at least one slit of the top layer configured to substantially align with the at least one slit on the bottom layer; and
bonding the layers together to sandwich the inner layer between the top and bottom layers thereby enclosing the fluidic channel, wherein the fluidic channel is positioned to allow diffusion communication between the fluidic channel and the slits.

19. The method of claim 18, wherein the top and bottom layers comprising a plurality of slits.

20. A method for making a device for islet transplantation comprising:
preparing a dissolvable mold configured to form an implantable device comprising:
a biocompatible frame configured to be inserted into tissue;
at least one slit configured to pass at least partially through the frame, wherein the at least one slit is sized and configured to promote vascular perfusion; and
a fluidic channel configured to retain cells and located within the biocompatible frame, the fluidic channel being separated from the at least one slit and able to communicate with the at least one slit through diffusion;
adding a polymerizable material to the mold;
polymerizing the material to form the implantable device; and
dissolving the mold.

21. The method of claim 20, wherein polymerizing the material comprises polymerizing the material with UV light.

22. The method of claim 20, wherein dissolving the mold comprises submerging the mold in a dissolving solution.

23. The method of claim 22, wherein the dissolving solution is citrus oil.

24. A method for treating diabetes comprising:
implanting the device of claim 1 into a tissue of a diabetic patient;
equilibrating the device within the tissue for a period of time sufficient to allow vascularization of the at least one slit; and
injecting a suspension of islet cells into the fluidic channel, wherein the islet cells secrete insulin into the at least one vascularized slit in response to glucose levels in the at least one vascularized slit.
